(19) United States
(12) Patent Application Publication
(10) Pub. No.: US 2023/0366876 A1
(43) Pub. Date: Nov. 16, 2023

(54) EXOSOME BASED ASSAYS FOR DETERMINING CANDIDATES FOR OSTEOARTHRITIS STEM CELL THERAPY

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, NIceville, FL (US)

(73) Assignee: CREATIVE MEDICAL TECHNOLOGIES, INC., Phoenix, AZ (US)

(21) Appl. No.: 18/315,364

(22) Filed: May 10, 2023

Related U.S. Application Data
(60) Provisional application No. 63/340,828, filed on May 11, 2022.

(51) Int. Cl. G01N 33/50; A61K 35/28

(52) U.S. Cl. CPC G01N 33/5044; A61K 35/28

(57) ABSTRACT

Disclosed are means and methods useful in selecting osteoarthritis patients most likely to respond to regenerative medicine therapy. One means of practicing the invention is to determine quantity and quality of inflammatory exosomes generated from the tissue adjacent to the injured cartilage. In another embodiment the invention provides detection of exosomes produced by inflammatory processes local or systemic to the body. In some embodiments therapies are tailored to the extent of inflammatory exosomes.

### CROSS-REFERENCE TO RELATED APPLICATIONS

This application claims priority to United States Provisional Application No. 63/340828, titled “Exosome Based Assays for Determining Candidates for Osteoarthritis Stem Cell Therapy” filed May 11, 2022, which is hereby incorporated by reference herein in its entirety.

### FIELD OF THE INVENTION

The invention relates to the analysis of exosomes for determining stem cell population candidates for regenerative therapies such as treating osteoarthritis.

### BACKGROUND OF THE INVENTION

The utilization of stem cell therapy in the area of osteoarthritis and cartilage degeneration offers the possibility of non-surgical induction of healing, a possibility that never was previously considered feasible in the medical field.

The administration of stem cells is believed to possess therapeutic activities mediated by the regenerative cells differentiating into de novo cartilage, as well as providing various antiinflammatory activities. There exist significant bodies of data that provide support for the possibility that stem cells should be clinically efficacious. Unfortunately, in many clinical studies, stem cells appear to possess positive activities at the preclinical level but when they enter human trials the cells work really well in some patients, but in some patients, there is no effect. In fact, in some cases, stem cells actually have been reported to endow a negative effect in the condition of the patient.

The heterogeneity of responses can be attributed to various host functions or activities. In some cases the inflammatory nature of the stem cell recipient acts to neutralize stem cells, or in some cases to even induce the stem cells to differentiate into scar tissue, thus eliciting negative responses instead of having a beneficial effect.

To date there are no consistent methods of selecting patients that would respond to stem cells before the actual stem cell administration.

One way in which the current invention addresses the lack of ability is to screen patients for stem cell therapy is to utilize biological markers. In some situations, biological markers are circulating proteins, peptides or small molecules. The current invention seeks to utilize exosomes as a marker of disease or inflammation which reduces efficacy of stem cell therapy.

Exosomes are small vesicles 40-100 nm in diameter, that are secreted by a number of different cell types for communicating with other cells via the proteins and ribonucleic acids they carry. An exosome is created intracellularly when a segment of the cell membrane spontaneously invaginates and is endocytosed. The internalized segment is broken into smaller vesicles that are subsequently expelled from the cell. The latter stage occurs when the late endosome, containing many small vesicles, fuses with the cell membrane, triggering the release of the vesicles from the cell. The vesicles (once released are called exosomes) consist of a lipid raft embedded with ligands common to the original cell membrane. Depending on their cellular origin, exosomes carry uniquely distinct profiles of proteins and/or nucleic acids (such as microRNAs (miRNAs)), which can trigger signaling pathways in other cells and/or transfer exosome products into other cells by exosome fusion with cellular plasma membranes. The protein composition of exosomes is distinct from that of other organelles, including early endosomes and plasma membranes, more closely resembling that of late endosomes or multivesicular bodies (MVBs). Exosomes are released from different cell types in varied physiological contexts. For example, B lymphocytes release exosomes carrying class II major histocompatibility complex molecules, which play a role in antigenic presentation. Similarly, dendritic cells produce exosomes (i.e., dexosomes, Dex), which play a role in immune response mediation, particularly in cytotoxic T lymphocyte stimulation. Some tumor cells secrete specific exosomes (i.e., texosomes, Tex) carrying tumor antigens in a regulated manner, which can present these antigens to antigen presenting cells. Exosomes may also carry pathogen-associated products. For example, exosomes have been known to carry products derived from Mycobacterium tuberculosis and Toxoplasma gondii-infected cells.

The invention seeks to utilize circulating exosomes as markers of inflammation in order to make the clinical decision if a patient should undergo stem cell therapy.

### SUMMARY

Preferred embodiments are directed to methods of assessing possibility of success in a stem cell procedure, said method comprising the steps of: a) selecting a patient suffering from a degenerative disease; b) obtaining a fluid from said patient; c) collecting exosomes from said fluid; and d) assessing said exosomes for molecules associated with resistance to stem cell therapy.

Preferred methods include embodiments wherein said stem cell procedure is administration of cells originating from a source comprising of: a) autologous; b) allogeneic; and c) xenogeneic.

Preferred methods include embodiments wherein said stem cells are derived from a source selected from a bone marrow source of origin.

Preferred methods include embodiments wherein said bone marrow aspirate source of origin stem cells are mononuclear cells which contain stem cells or unmanipulated.

Preferred methods include embodiments in which said mononuclear cells contain endothelial progenitor cells.

Preferred methods include embodiments in which said endothelial progenitor cells possess the marker CD133.

Preferred methods include embodiments in which said endothelial progenitor cells possess the marker CD33.

Preferred methods include embodiments in which said endothelial progenitor cells possess the marker CD34.

Preferred methods include embodiments in which said endothelial progenitor cells possess the marker c-kit.

Preferred methods include embodiments in which said endothelial progenitor cells possess the marker interleukin-3 receptor.

Preferred methods include embodiments in which said endothelial progenitor cells possess the marker c-maf.

Preferred methods include embodiments in which said endothelial progenitor cells possess the marker IL-6 receptor.

Preferred methods include embodiments in which said endothelial progenitor cells possess the marker Steel Factor receptor.

Preferred methods include embodiments in which said endothelial progenitor cells possess the IL-12 receptor.

Preferred methods include embodiments in which said endothelial progenitor cells possess the marker IL-17 receptor.

Preferred methods include embodiments in which said endothelial progenitor cells possess the marker angiopoietin receptor.

Preferred methods include embodiments in which said endothelial progenitor cells possess the marker thrombopoietin receptor.

Preferred methods include embodiments in which said endothelial progenitor cells possess the marker PDGF-BB receptor.

Preferred methods include embodiments in which said endothelial progenitor cells possess the marker EGF receptor.

Preferred methods include embodiments in which said endothelial progenitor cells possess the marker FGF-1 receptor.

Preferred methods include embodiments in which said endothelial progenitor cells possess the marker FGF-2 receptor.

Preferred methods include embodiments in which said endothelial progenitor cells possess the marker CD133.

Preferred methods include embodiments in which said mononuclear cells contain chondrocytic progenitor cells.

Preferred methods include embodiments in which said mononuclear cells contain mesenchymal stem cells.

Preferred methods include embodiments wherein said mesenchymal stem cells express vimentin.

Preferred methods include embodiments wherein said mesenchymal stem cells express CD29.

Preferred methods include embodiments wherein said mesenchymal stem cells express CD36.

Preferred methods include embodiments wherein said mesenchymal stem cells express CD37.

Preferred methods include embodiments wherein said mesenchymal stem cells express CD73.

Preferred methods include embodiments wherein said mesenchymal stem cells express CD90.

Preferred methods include embodiments wherein said mesenchymal stem cells express CD166.

Preferred methods include embodiments wherein said mesenchymal stem cells express SSEA4.

Preferred methods include embodiments wherein said mesenchymal stem cells express CD9.

Preferred methods include embodiments wherein said mesenchymal stem cells express CD44.

Preferred methods include embodiments wherein said mesenchymal stem cells express CD146.

Preferred methods include embodiments wherein said mesenchymal stem cells express CD105.

Preferred methods include embodiments wherein said mesenchymal stem cells express HLA-G.

Preferred methods include embodiments wherein said mesenchymal stem cells express CD37.

Preferred methods include embodiments wherein said bone marrow mononuclear cells possess ability to generate soluble TNF-alpha receptor at a concentration of 10 pg-1 ng per 1,000,000 cells under basal growth conditions in DMEM media with 10% fetal calf serum.

Preferred methods include embodiments wherein said cells possess ability to generate soluble TNF-alpha receptor at a concentration of 50 pg-500 pg per 1,000,000 cells under basal growth conditions in DMEM media with 10% fetal calf serum.

Preferred methods include embodiments wherein said cells possess ability to generate soluble HLA-G receptor at a concentration of 1 pg-10 ng per 1,000,000 cells under basal growth conditions in DMEM media with 10% fetal calf serum.

Preferred methods include embodiments wherein said cells possess ability to generate soluble HLA-G receptor at a concentration of 10 pg-1 ng per 1,000,000 cells under basal growth conditions in DMEM media with 10% fetal calf serum.

Preferred methods include embodiments wherein said cells possess ability to generate soluble HLA-G at a concentration of 50 pg-500 ng per 1,000,000 cells under basal growth conditions in DMEM media with 10% fetal calf serum or platelet lysate liquid or lyophilized.

Preferred methods include embodiments wherein said cells possess ability to generate soluble HLA-G at a concentration of 10 pg-1 ng per 1,000,000 cells under basal growth conditions in DMEM media with 10% fetal calf serum or platelet lysate liquid or lyophilized.

Preferred methods include embodiments wherein said cells possess ability to generate interleukin 10 at a concentration of 1 pg-10 ng per 1,000,000 cells under basal growth conditions in DMEM media with 10% fetal calf serum or platelet lysate liquid or lyophilized.

Preferred methods include embodiments wherein said cells possess ability to generate interleukin 10 at a concentration of 10 pg-1 ng per 1,000,000 cells under basal growth conditions in DMEM media with 10% fetal calf serum or platelet lysate liquid or lyophilized.

Preferred methods include embodiments wherein said cells possess ability to generate interleukin 10 at a concentration of 50 pg-500 ng per 1,000,000 cells under basal growth conditions in DMEM media with 10% fetal calf serum or platelet lysate liquid or lyophilized.

Preferred methods include embodiments wherein said cells possess ability to generate interleukin 10 at a concentration of 50 pg-200 ng per 1,000,000 cells under basal growth conditions in DMEM media with 10% fetal calf serum or platelet lysate liquid or lyophilized.

Preferred methods include embodiments wherein said cells possess ability to generate interleukin 35 at a concentration of 1 pg-10 ng per 1,000,000 cells under basal growth conditions in DMEM media with 10% fetal calf serum or platelet lysate liquid or lyophilized.

Preferred methods include embodiments wherein said cells possess ability to generate interleukin 35 at a concentration of 10 pg-1 ng per 1,000,000 cells under basal growth conditions in DMEM media with 10% fetal calf serum or platelet lysate liquid or lyophilized.

Preferred methods include embodiments wherein said cells possess ability to generate interleukin 35 at a concentration of 50 pg-500 ng per 1,000,000 cells under basal growth conditions in DMEM media with 10% fetal calf serum or platelet lysate liquid or lyophilized.

Preferred methods include embodiments wherein said cells possess ability to generate interleukin 35 at a concentration of 50 pg-200 ng per 1,000,000 cells under basal growth conditions in DMEM media with 10% fetal calf serum or platelet lysate liquid or lyophilized.

Preferred methods include embodiments wherein said cells possess ability to generate interleukin 4 at a concentration of 1 pg-10 ng per 1,000,000 cells under basal growth conditions in DMEM media with 10% fetal calf serum or platelet lysate liquid or lyophilized.

Preferred methods include embodiments wherein said cells possess ability to generate interleukin 4 at a concentration of 10 pg-1 ng per 1,000,000 cells under basal growth conditions in DMEM media with 10% fetal calf serum or platelet lysate liquid or lyophilized.

Preferred methods include embodiments wherein said cells possess ability to generate interleukin 4 at a concentration of 50 pg-500 ng per 1,000,000 cells under basal growth conditions in DMEM media with 10% fetal calf serum or platelet lysate liquid or lyophilized.

Preferred methods include embodiments wherein said cells are isolated by selection for markers selected from a group comprising of: CD39, CD73, FOXP3, GITR, CLTA4, ICOS, GARP, LAP, PD-1, CCR6, and CXCR3.

Preferred methods include embodiments wherein said cell population expresses OCT-4.

Preferred methods include embodiments wherein said cell population expresses SOX-2.

Preferred methods include embodiments wherein said cell population expresses NANOG.

Preferred methods include embodiments wherein said cell population expresses c-Met.

Preferred methods include embodiments wherein said cell population expresses PDGF receptor.

Preferred methods include embodiments wherein said cell population expresses OCT-4.

Preferred methods include embodiments wherein said cell population expresses CD13 and CD73.

Preferred methods include embodiments wherein said cell population expresses CD29 and CD73.

Preferred methods include embodiments wherein said cell population expresses CD54 and CD73.

Preferred methods include embodiments wherein said cell population expresses SSEA4 and CD73.

Preferred methods include embodiments wherein said cell population expresses CD31 and CD73.

Preferred methods include embodiments wherein said cell population expresses CD34 and CD73.

Preferred methods include embodiments wherein said cell population expresses TNF-alpha receptor p55 and CD73.

Preferred methods include embodiments wherein said cell population expresses TNF-alpha receptor p75 and CD73.

Preferred methods include embodiments wherein said cell population expresses interleukin-1 beta receptor and CD73.

Preferred methods include embodiments wherein said cell population expresses interleukin-6 receptor and CD73.

Preferred methods include embodiments wherein said cell population expresses interleukin-8 receptor and CD73.

Preferred methods include embodiments wherein said cell population expresses interleukin-11 receptor and CD73.

Preferred methods include embodiments wherein said cell population expresses complement receptor-2 and CD73.

Preferred methods include embodiments wherein said cell population expresses complement receptor-3 and CD73.

Preferred methods include embodiments wherein said cell population expresses complement receptor-4 and CD73.

Preferred methods include embodiments wherein exosomes are collected using ultracentrifugation.

Preferred methods include embodiments wherein said ultracentrifugation utilizing a density gradient to collect exosomes.

Preferred methods include embodiments wherein said density gradient is comprised of cesium chloride.

Preferred methods include embodiments wherein said exosomes are collected by an affinity selection means, wherein said exosomes possess an increased affinity to a solid surface as compared to other materials.

Preferred methods include embodiments wherein said exosomes bind to said solid surface do to expression of fas ligand on the exosome.

Preferred methods include embodiments wherein said exosomes bind to said solid surface due to expression of CD9 on the exosome.

Preferred methods include embodiments wherein said exosomes bind to said solid surface due to expression of tetraspanin on the exosome.

Preferred methods include embodiments wherein said exosomes bind to said solid surface due to expression of CD1a on the exosome.

Preferred methods include embodiments wherein said exosomes bind to said solid surface due to expression of CD11c on the exosome.

Preferred methods include embodiments wherein said exosomes bind to said solid surface due to expression of annexin-v on the exosome.

Preferred methods include embodiments wherein said exosomes bind to said solid surface due to expression of calreticulin on the exosome.

Preferred methods include embodiments wherein said exosomes bind to said solid surface due to expression of hsp-20 on the exosome.

Preferred methods include embodiments wherein said exosomes bind to said solid surface due to expression of hsp-70 on the exosome.

Preferred methods include embodiments wherein said exosomes bind to said solid surface due to expression of hsp-76 on the exosome.

Preferred methods include embodiments wherein said exosomes bind to said solid surface due to expression of hsp-90 on the exosome.

Preferred methods include embodiments wherein said exosomes bind to said solid surface due to expression of MHC I on the exosome.

Preferred methods include embodiments wherein said exosomes bind to said solid surface due to expression of CD80 on the exosome.

Preferred methods include embodiments wherein said exosomes bind to said solid surface due to expression of CD86 on the exosome.

Preferred methods include embodiments wherein exosomes expression of miRNA-55 represents poor candidates for stem cell administration.

Preferred methods include embodiments wherein exosomes expression of miRNA-129 represents poor candidates for stem cell administration.

Preferred methods include embodiments wherein exosomes are concentrated based on their affinity to a lectin.

Preferred methods include embodiments wherein said lectin is lens culinaris agglutin (LCA).

Preferred methods include embodiments wherein said lectin is Lens culinaris lectin (LCH).

Preferred methods include embodiments wherein said lectin is Galanthus nivalis lectin (GNA).

Preferred methods include embodiments wherein said lectin is Narcissus pseudonarcissus lectin (NPL).

Preferred methods include embodiments wherein said lectin is Allium sativum lectin (ASA).

Preferred methods include embodiments wherein said lectin is Sambucus nigra lectin (SNA).

Preferred methods include embodiments wherein said lectin is Maackia amurensis lectin (MAL).

Preferred methods include embodiments wherein said lectin is Concanavalin A (Con A).

Preferred methods include embodiments wherein said lectin is Aleuria aurantia lectin (AAL).

Preferred methods include embodiments wherein said lectin is Lotus tetragonolobus lectin (LTL).

Preferred methods include embodiments wherein said lectin is Naja mossambica lectin (NML).

Preferred methods include embodiments wherein said lectin is Dolichos biflorus agglutinin (DBA).

Preferred methods include embodiments wherein said lectin is Helix aspersa lectin (HAL).

Preferred methods include embodiments wherein said lectin is Psophocarpus tetragonolobus lectin II (PTL II).

Preferred methods include embodiments wherein said lectin is Wisteria floribunda lectin (WFL).

Preferred methods include embodiments wherein said lectin is Erythrina cristagalli lectin (ECL).

Preferred methods include embodiments wherein said lectin is Griffonia simplicifolia lectin II (GSL II).

Preferred methods include embodiments wherein said lectin is Phaseolus vulgaris leucoagglutinin (PHA-L).

Preferred methods include embodiments wherein said lectin is Phytohemagglutinin (PHA).

### DETAILED DESCRIPTION OF THE INVENTION

The current invention utilizes exosomes as a means of assessing possibility of patients recovering as a result of stem cell therapy. More specifically, the invention utilizes the quantification of exosomes as a biological marker to assess extent of various inflammatory process, wherein said inflammatory processes increase the risk of failure of stem cell therapy. The invention aims to identify efficacy of stem cell therapy in the area of osteoarthritis.

As used herein, the terms “biomarker” and “infectious agent-associated biomarker” are used interchangeably with reference to any molecular entity that can be used as an indicator of a disease, including an acute infectious disease or chronic infectious disease condition in an organism. The biomarker may be any detectable protein, nucleic acid, such as an mRNA or microRNA, lipid, or any product present and/or differentially expressed in exosomes present in bodily fluids following an infection and/or coincident with an infectious disease condition whose presence and/or concentration reflects the presence, severity, type or progression of an acute or chronic infection in a subject. In molecular terms, biomarkers may be detected and quantitated in a subject using genomics, proteomics technologies or imaging technologies.

An “inflammation associated biomarker” or “virus-associated cellular biomarker” are used with reference to cellular biomarkers whose expression is altered in response to a disease associated (such as a virus) or infectious disease condition and whose differential expression relative to non-infected cells is diagnostic of an infection or disease caused by that particular infectious agent.

The term “antibodies” as used herein includes native antibodies, as well as any antibody derived fragment selected from the group consisting of: IgG, antibody variable region; isolated CDR region; single chain Fv molecule (scFv) comprising a VH and VL domain linked by a peptide linker allowing for association between the two domains to form an antigen binding site; bispecific scFv dimer; minibody comprising a scFv joined to a CH3 domain; diabody (dAb) fragment; single chain dAb fragment consisting of a VH or a VL domain; Fab fragment consisting of VL, VH, CL and CHI domains; Fab′ fragment, which differs from a Fab fragment by the addition of a few residues at the carboxyl terminus of the heavy chain CHI domain, including one or more cysteines from the antibody hinge region; Fab′-SH fragment, a Fab′ fragment in which the cysteine residue(s) of the constant domains bear a free thiol group; F(ab′).sub.2, bivalent fragment comprising two linked Fab fragments; Fd fragment consisting of VH and CHI domains; derivatives thereof; and any other antibody fragment(s) retaining antigen-binding function. Fv, scFv, or diabody molecules may be stabilized by the incorporation of disulphide bridges linking the VH and VL domains. When using antibody-derived fragments, any or all of the targeting domains therein and/or Fc regions may be “humanized” using methodologies well known to those of skill in the art. In some embodiments, the infectious-agent associated antibody is modified to remove the Fc region.

As used herein, the term “specifically binding” refers to the interaction between binding pairs (e.g., an antibody and its target antigen or peptide, or between two nucleic acids), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.). In some embodiments, the interaction has an equilibrium dissociation constant (Kd) of at least 10.sup.-6 moles/liter, at least 10.sup.-7 moles/liter, at least 10.sup.-8 moles/liter or at least 10.sup.-9 moles/liter.

As used herein, the term “bodily fluid sample” refers to a sample of bodily fluid obtained from a mammal subject, preferably a human subject. Exemplary bodily fluid samples include urine, blood, saliva, serum, plasma, cyst fluid, pleural fluid, ascites fluid, peritoneal fluid, amniotic fluid, epididymal fluid, cerebrospinal fluid, bronchoalveolar lavage fluid, breast milk, tears, sputum, and combinations thereof. In a preferred embodiment, the bodily fluid sample is urine. Unless otherwise noted, as used herein, the terms “bodily fluid sample” and “sample” are to be considered synonymous with any of the above-described bodily fluid samples.

As used herein, a “detectable label” refers to a molecule, compound or a group of molecules and/or compounds associated with detection of a polypeptide biomarker or nucleic acid biomarker. Signals from the detectable label may be detected by various means depending on the nature of the detectable label. Detectable labels may include radioisotopes, fluorescent compounds (e.g., fluorescein, rhodamine, phycoerythrin, chemiluminescent compounds, bioluminescent compounds, nucleotide chromophores, substrates, enzymes (e.g., alkaline phosphatase, horseradish peroxidase, .beta.-glactosidase, luciferase, green fluorescent protein (GFP), blue fluorescent protein (BFP)), enzyme substrates (e.g., fluorescent moieties or proteins, luminescent moieties (e.g., luciferin, aequorin, Evidot.RTM. quantum dots supplied by Evident Technologies, Troy, N.Y., or Qdot.TM. nanoparticles supplied by the Quantum Dot Corporation, Palo Alto, Calif.), colorimetric labels, such as colloidal gold, colored glass or plastic (e.g., polystyrene, polypropylene, and latex) beads and the like. Exemplary means for detecting a detectable label include but are not limited to spectroscopic, photochemical, biochemical, immunochemical, electrical, electromagnetic, radiochemical, optical or chemical means and combinations thereof, and may encompass fluorescence, chemifluorescence, chemiluminescence, electrochemiluminescence and the like. A detectable label can be attached or structurally incorporated into a detection antibody or a secondary antibody.

As used herein, a “biomarker profile” refers to one or more biomarkers diagnostic for a particular disease and/or infection. The biomarker profile may include biomarkers directly derived from a cellular gene or infectious agent gene, including those whose expression levels or profile is characteristic of an individual who has a disease or is acutely or chronically infected with a particular infectious agent. Accordingly, the step of determining whether the subject carries a disease associated biomarker may be based on detecting the presence, absence or differential expression of one or more disease-associated biomarkers present in the isolated exosomes. As used herein, the term “differential expression” refers to a qualitative and/or quantitative changes in biomarker expression levels relative to a control sample.

The term “increased level” refers to an expression level that is higher than a normal or control level customarily defined or used in the relevant art. For example, an increased level of immunostaining of an exosome preparation from a bodily fluid sample is a level of immunostaining that would be considered higher than the level of immunostaining of a control exosome preparation by a person of ordinary skill in the art. As used herein, the described biomarker may exhibit increased expression levels of at least 10%, at least 20%, at least 30%, at least 40%, at least 50%, at least 80%, at least 100%, at least 2-fold, at least 3-fold, at least 5-fold, at least 10-fold, at least 50-fold or at least 100-fold increase or more relative to a suitable reference level.

The term “decreased level” refers to an expression level that is lower than a normal or control level customarily defined or used in the relevant art. As used herein, the described biomarkers may exhibit decreased expression levels of at least at least 10%, at least 20%, at least 30%, at least 40%, at least 50%, at least 80%, at least 100%, at least 2-fold, at least 3-fold, at least 5-fold, at least 10-fold, at least 50-fold or at least 100-fold decrease or more relative to a suitable reference level.

The term “expression level of a disease-associated biomarker” may be measured at the transcription level, in which case the presence and/or the amount of a polynucleotide is determined, or at the translation level, in which case the presence and/or the amount of a polypeptide is determined.

The terms “gene product” and “expression product of a gene” refers to the transcriptional products of a gene, such as mRNAs and cDNAs encoded by the gene, and/or the translational products of a gene, such as peptides encoded by the gene, and fragments thereof.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

In one embodiment of the invention, a patient suffering from osteoarthritis is assessed for inflammation using exosomes isolated from a variety of bodily tissues and/or fluids. In some cases the exosomes are separated using convention means such as magnetic activated cell sorting or ExoFlo type approaches that purified exosomes based on affinity means.

In some embodiments exosomes may be concentrated by antibodies to various exosome associated markers present on exosomes. As described previously, asparagine-linked glycosylation 6 homolog (ALG6) is an alpha-1,3-glucosyltransferase which catalyzes the addition of glucose residue to the lipid-linked oligosaccharide precursor for N-linked glycosylation. Although, the enzyme is localized to the endoplasmic reticulum of human cells, antibodies to an ALG6 peptide were unexpectedly found to exhibit unique exosome capture capabilities. In one aspect, a composition for exosome capture comprising an anti-ALG6 antibody. The antibody may specifically bind to human ALG6 or other mammalian homologs thereof. In one embodiment, the anti-ALG6 antibody specifically binds a human ALG6 peptide consisting. As such, the exosome capture antibody may be specifically raised against an immunogenic composition comprising a peptide consisting of the amino acid sequence. In some embodiments, the exosome capture antibody is a polyclonal anti-ALG6 antibody. In other embodiments, the exosome capture antibody is a monoclonal anti-ALG6 antibody. In another embodiment, an anti-ALG6 antibody directed against a synthetic peptide corresponding to a region within the N terminal amino acids 38-50 (GDYEAQRHWQEIT) of Human ALG6 (alpha-1,3-glucosyltransferase). Other exosome specific compounds include Annexin-V, calreticulin, and CD9. In some embodiments, a detectable label or binding moiety, such as biotin, is conjugated or structurally incorporated into the antibody targeting the exosome-specific compound. The addition of binding moieties, such as biotin, can facilitate conjugation to other macromolecules, such as streptavidin-linked enzymes, such as streptavidin-HRP or streptavidin-linked secondary antibodies for detection of exosome biomarkers as further described below. In other embodiments, the composition includes an antibody attached or immobilized onto a solid substrate, such as a microtiter plate, for capturing exosomes. This can allow for in situ interrogation disease associated biomarkers in exosomes bound to the solid substrate. In some embodiments, two or more antibodies may be immobilized onto the solid substrate, each binding to different epitopes on an exosome protein and/or different exosome proteins. The antibodies may include both polyclonal antibodies and monoclonal antibodies and may include native immunoglobulin molecules, as well as fragments and/or combinations of those immunoglobulin molecules, including humanized versions of immunoglobulin molecules or fragments thereof, as long as they retain the ability to serve as exosome capture agents or detection agents for disease associated biomarkers. In some cases, as an alternative to antibodies, lectins may be used alone or in combination with the anti-ALG6 antibodies. Lectins have a particular affinity for glycan markers, such as glycoproteins, which are often present in exosomes derived from cartilage tissues. Non-limiting examples of lectins for immobilization on a substrate include Lens culinaris agglutin (LCA), Lens culinaris lectin (LCH), Galanthus nivalis lectin (GNA), Narcissus pseudonarcissus lectin (NPL), Allium sativum lectin (ASA), Sambucus nigra lectin (SNA), Maackia amurensis lectin (MAL), Concanavalin A (Con A), Aleuria aurantia lectin (AAL), Lotus tetragonolobus lectin (LTL), Naja mossambica lectin (NML), Dolichos biflorus agglutinin (DBA), Helix aspersa lectin (HAL), Psophocarpus tetragonolobus lectin II (PTL II), Wisteria floribunda lectin (WFL), Erythrina cristagalli lectin (ECL), Griffonia simplicifolia lectin II (GSL II) and Phaseolus vulgaris leucoagglutinin (PHA-L). In some embodiments detection of miRNA-155 is used to predict poor response to stem cell therapy. In certain embodiments, exosomes are bound to a solid substrate with immobilized lectins binding high mannose structures, including 1,3- or 1,6-linked high mannose structures on the surface of exosomes. In one embodiment, Galanthus nivalis (GNA) lectins are bound to a solid substrate. GNA lectins can bind to 1,3 and 1,6-linked high mannose structures on the surface of exosomes. The solid substrate to which the capture antibody is bound may be any water-insoluble or water-insuspendable solid substrate. Exemplary solid substrates encompassed herein include microtiter plates made of plastics, such as polystyrene and polypropylene, those formed partially or entirely of glass (e.g., controlled pore glass), polysaccharides (e.g., agarose), polyacrylamides, silicones, plastics, such as polystyrene, polypropylene and polyvinyl alcohol, solid and semi-solid matrixes, such as aerogels and hydrogels, resins, beads, magnetic beads, biochips (including thin film coated biochips), nanoparticles, microfluidic chips, a silicon chips, multi-well plates (also referred to as micro-titer plates or microplates), membranes, filters, conducting and non-conducting metals, glass (including microscope slides) and magnetic supports, including magnetizable particles of cellulose or other polymers. Further examples of solid substrates include silica gels, polymeric membranes, particles, derivatized plastic films, glass beads, cotton, plastic beads, alumina gels, and polysaccharides such as Sepharose, nylon, latex bead, magnetic bead, paramagnetic bead, super-paramagnetic bead, starch and the like.

Exosome capture agents, including CD68 antibodies ALG6 antibodies, CD6 antibodies, CD8 antibodies, CD9 antibodies, may be bound to the solid substrate by covalent bonds or by adsorption. The capture agents may be attached using any crosslinking agents suitable for attachment of antibodies. Reagents included in the methods and kits described herein may be provided as reagents embedded, linked, connected, attached, placed or fused to any of the solid substrate materials described above.

In the practice of the invention, in some embodiments, sample fluids may be passed through a centrifuge filter for isolating and concentrating exosomes prior to immobilization onto the solid substrate as further described below. This may further help to reduce non-specific binding of sample fluid components to the solid substrate. Alternatively, exosomes may be purified by affinity (or column) chromatography using anti-ALG6 antibodies immobilized on a suitable column matrix, whereby a bodily fluids are loaded onto the column enabling exosomes to bind to the anti-ALG6 antibodies in a first step and then elute the exosomes bound to the column in a second step. The eluted exosomes may then be tested directly for biomarker binding or they may be applied to the above described solid substrate (i.e., a second solid substrate).

Those of ordinary skill in the art are aware of various methods and devices available in the measurement and analysis of exosomes and/or associated compounds. For polypeptides or proteins contained in samples of patients, immunoassay devices and methods are often used. These devices and methods may generate a signal related to the presence or amount of an analyte of interest by using labeled molecules in various sandwiches and competitive or non-competitive assay formats. Additionally, the presence or amount of an analyte may be determined without the need for labeled molecules using certain methods and devices, such as biosensors and optical immunoassays. Although other methods (e.g., measurement of marker RNA level) are well known to those skilled in the art, immunoassay is preferably used for the measurement. The presence or amounts of markers are generally identified by detecting specific binding using an antibody specific for each marker. Any suitable immunoassay, e.g., enzyme-linked immunosorbent assay (ELISA), radioimmunoassay (RIA), competitive binding assay, and planar waveguide technique, may be used. Specific immunological binding of the antibody to a marker may be detected directly or indirectly. Direct labels include fluorescent or luminescent tags, metals, dyes, and radionuclides attached to the antibody. Indirect labels include various enzymes well known in the art, e.g., alkaline phosphatase and horseradish peroxidase. When the sample is blood, it may be combined with various components after collection to preserve or prepare the sample for subsequent techniques. For example, the blood is treated with an anticoagulant, a cell fixative, a protease inhibitor, a phosphatase inhibitor, a protein, or a DNA, or a RNA preservative after collection. For example, blood is collected via venipuncture using vacuum collection tubes containing an anticoagulant such as EDTA or heparin. Blood may also be collected using a heparin-coated syringe and hypodermic needle. Blood may also be combined with components that will be useful for cell culture. For example, blood may be combined with a cell culture medium or a supplemented cell culture medium (e.g., cytokines). Also, when the sample is blood, it is advantageous that the brain-derived vesicles may be isolated by using vesicles freely passing through the blood brain barriers without collecting brain tissue.

1. A method of assessing possibility of success in a stem cell procedure, said method comprising the steps of: a) selecting a patient suffering from a degenerative disease; b) obtaining a fluid from said patient; c) collecting exosomes from said fluid; and d) assessing said exosomes for molecules associated with resistance to stem cell therapy.

2. The method of claim 1, wherein said stem cell procedure is administration of cells originating from a source comprising of: a) autologous; b) allogeneic; and c) xenogeneic.

3. The method of claim 1, wherein said stem cells are derived from a source selected from a bone marrow source of origin.

4. The method of claim 3, wherein said bone marrow aspirate source of origin stem cells are mononuclear cells which contain stem cells or unmanipulated cells.

5. The method of claim 4, in which said mononuclear cells contain endothelial progenitor cells.

6. The method of claim 5, in which said endothelial progenitor cells possess the marker interleukin-3 receptor.

7. The method of claim 5, in which said endothelial progenitor cells possess the marker c-maf.

8. The method of claim 5, in which said endothelial progenitor cells possess the marker IL-6 receptor.

9. The method of claim 5, in which said endothelial progenitor cells possess the marker Steel Factor receptor.

10. The method of claim 5, in which said endothelial progenitor cells possess the IL-12 receptor.

11. The method of claim 5, in which said endothelial progenitor cells possess the marker IL-17 receptor.

12. The method of claim 5, in which said endothelial progenitor cells possess the marker angiopoietin receptor.

13. The method of claim 5, in which said endothelial progenitor cells possess the marker thrombopoietin receptor.

14. The method of claim 5, in which said endothelial progenitor cells possess the marker PDGF-BB receptor.

15. The method of claim 3, wherein said bone marrow mononuclear cells possess ability to generate soluble TNF-alpha receptor at a concentration of 10 pg-1 ng per 1,000,000 cells under basal growth conditions in DMEM media with 10% fetal calf serum.

16. The method of claim 3, wherein said cells are isolated by selection for markers selected from a group comprising of: CD39, CD73, FOXP3, GITR, CLTA4, ICOS, GARP, LAP, PD-1, CCR6, and CXCR3.

17. The method of claim 1, wherein said exosomes are collected by an affinity selection means, wherein said exosomes possess an increased affinity to a solid surface as compared to other materials.

18. The method of claim 1, wherein exosomes expression of miRNA-55 represents poor candidates for stem cell administration.

19. The method of claim 1, wherein exosomes expression of miRNA-129 represents poor candidates for stem cell administration.

20. The method of claim 1, wherein exosomes are concentrated based on their affinity to a lectin.
